(19) United States
(12) Patent Application Publication
(10) Pub. No.: US 2026/0199385 A1
(43) Pub. Date: Jul. 16, 2026

(54) USE OF GPPNHP IN PREPARATION OF MEDICAMENT FOR TREATING SYSTEMIC LUPUS ERYTHEMATOSUS

(71) Applicant: BEIJING HOSPITAL, Beijing (CN)

(72) Inventors: Xuan ZHANG, Beijing (CN); Wei Liu, Beijing (CN); Yi Yin, Beijing (CN); Shiliang Ma, Beijing (CN)

(73) Assignee: BEIJING HOSPITAL, Beijing (CN)

(21) Appl. No.: 19/420,863

(22) Filed: Dec. 16, 2025

Related U.S. Application Data
(63) Continuation of application No. PCT/CN2025/108736, filed on Jul. 16, 2025.

(30) Foreign Application Priority Data
Jan. 13, 2025 (CN) 202510045665.2

(51) Int. Cl. A61K 31/708; A61K 47/02; A61P 37/06

(52) U.S. Cl. CPC A61K 31/708; A61K 47/02; A61P 37/06

(57) ABSTRACT

With the use of necroptosis inhibitor GppNHp, the significant alleviation of multiple symptoms of lupus model mice is achieved, such as significant reduction of spleen volume and spleen weight, significant reduction of lymph node volume and lymph node weight, mitigation of kidney injury, reduction of urinary protein, reduction of tumor necrosis factor-α (TNF-α) concentration, and restoration of complement C3 concentration. A formulation can effective improve symptoms of lupus mice, and is expected to improve quality of life of SLE patients.

### CROSS REFERENCE TO THE RELATED APPLICATIONS

This application is a continuation application of International Application No. PCT/CN2025/108736, filed on July 16, 2025, which is based upon and claims priority to Chinese Patent Application No. 202510045665.2, filed on January 13, 2025, the entire contents of which are incorporated herein by reference.

### TECHNICAL FIELD

The present invention belongs to the field of medical technologies, and particularly relates to use of GppNHp in preparation of a medicament for treating systemic lupus erythematosus.

### BACKGROUND

Systemic lupus erythematosus (SLE) is a complex autoimmune disease and occurs frequently in young women, and a pathological mechanism mainly involves overactivated B-cell signaling and T-cell signaling and abnormal release of cytokines. At present, treatment of SLE mainly relies on widely used nonspecific hormones and immunosuppressants. These treatment methods enable control of a condition and alleviation of symptoms to a certain extent, but long-term use causes a series of side effects, such as osteoporosis, increased infection risk, and kidney injury.

Existing treatment methods mainly includes: 1. Glucocorticoids: used for rapidly controlling inflammatory responses, but long-term use causes severe side effects. 2. Antimalarial drugs (such as hydroxychloroquine): used for treating mild-to-moderate SLE and providing immunomodulatory effects; however, treatment effects are limited. 3. Immunosuppressants (such as cyclophosphamide): used for treating severe SLE, but possibly causing excessive suppression of an immune system and other severe side effects. 4. Biologics (such as belimumab): targeting specific immune cells or cytokines, and although treatment efficacy is significant, cost is high and side effects such as infection possibly occur. A common problem of these treatment regimens is that these treatment regimens cannot fundamentally regulate or correct immune abnormalities causing SLE and are accompanied by severe side effects, which limit long-term use.

Although existing treatment methods play a role in controlling SLE symptoms, significant insufficiency exists in safety, persistence of efficacy, and cost-effectiveness. Particularly, long-term dependence on hormones and immunosuppressants not only causes multiple complications but also results in gradually weakened treatment effects for a portion of patients. There is an urgent need to develop a novel treatment method for more safely and more effectively regulating an immune system of patients, reducing side effects caused by long-term treatment, and improving quality of life of patients. Necroptosis is a regulated cell-death form and has been found in recent years to be closely associated with pathogenesis of various autoimmune diseases. Therefore, research of a necroptosis inhibitor as an intervention medicament for treating SLE is particularly important.

### SUMMARY

In view of the foregoing, an objective of the present invention is to provide use of GppNHp in preparation of a medicament for treating systemic lupus erythematosus. With the use of necroptosis inhibitor GppNHp, the present invention enables significant alleviation of multiple symptoms of lupus model mice, including reduction of spleen volume and spleen weight, reduction of lymph node volume and lymph node weight, mitigation of kidney injury, reduction of urinary protein concentration, and regulation of related immune-inflammatory response indices. The present invention provides a safe and effective formulation and is expected to improve quality of life of patients.

To achieve the foregoing objective, the present invention provides the following technical solutions.

The present invention provides use of GppNHp in preparation of a medicament for treating systemic lupus erythematosus.

Preferably, a concentration of GppNHp is 5-15 μg/μL.

Preferably, a solvent of GppNHp is phosphate buffer.

Preferably, GppNHp is used in injection.

Preferably, GppNHp is used in a frequency of 1-5 days/time.

The present invention further provides a medicament for treating systemic lupus erythematosus, where the medicament includes GppNHp and a pharmaceutically acceptable carrier thereof.

Compared with the prior art, the present invention has the following beneficial effects: The present invention provides use of GppNHp in preparation of a medicament for treating systemic lupus erythematosus. With the use of necroptosis inhibitor GppNHp, the present invention enables significant alleviation of multiple symptoms of SLE, including significant reduction of spleen volume and spleen weight, reduction of lymph node volume and lymph node weight, mitigation of kidney injury, reduction of urinary protein and inflammatory factor (tumor necrosis factor-α (TNF-α)) concentration, and restoration of complement C3 concentration. A formulation provided by the present invention can effective improve symptoms of lupus mice, and is expected to improve quality of life of SLE patients.

### BRIEF DESCRIPTION OF THE DRAWINGS

FIGS. 1A-1D show results of spleen and axillary lymph node volume and weight of mice before and after treatment with necroptosis inhibitor GppNHp, where FIG. 1A shows spleen volume of mice before and after treatment with necroptosis inhibitor GppNHp, FIG. 1B shows spleen weight of mice before and after treatment with necroptosis inhibitor GppNHp, FIG. 1C shows axillary lymph node volume of mice before and after treatment with necroptosis inhibitor GppNHp, and FIG. 1D shows axillary lymph node weight of mice before and after treatment with necroptosis inhibitor GppNHp.

FIG. 2 shows kidney pathological HE-stained images of mice before and after treatment with necroptosis inhibitor GppNHp, with a scale bar of 20 μm.

FIGS. 3A-3C show changes in autoantibodies (anti-dsDNA antibody), complement C3, and tumor necrosis factor TNF-α levels of mice before and after treatment with necroptosis inhibitor GppNHp.

FIGS. 4A-4B show changes in blood creatinine and urinary protein concentration of mice before and after treatment with necroptosis inhibitor GppNHp.

FIG. 5 shows viability of peripheral blood neutrophils of mice detected by flow cytometry before and after treatment.

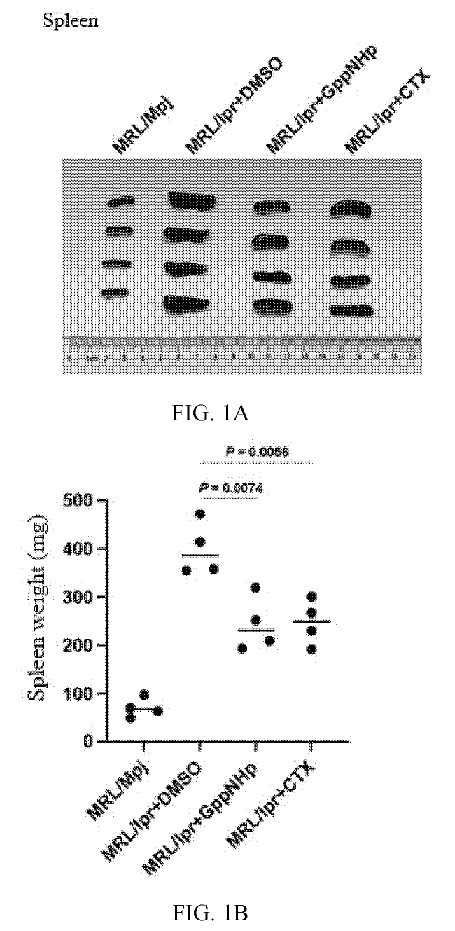

In FIGS. 1A-5, MRL/Mpj indicates a normal mouse group, MRL/lpr+DMSO indicates an untreated lupus model mouse group using DMSO as control, MRL/lpr+GppNHp indicates a lupus model mouse group treated with necroptosis inhibitor GppNHp, and MRL/lpr+CTX indicates a lupus model mouse group treated with clinical first-line drug cyclophosphamide (CTX).

### DETAILED DESCRIPTION OF THE EMBODIMENTS

The present invention provides use of set for GppNHp in preparation of a medicament for treating systemic lupus erythematosus.

In the present invention, GppNHp is dissolved in phosphate buffer and sterilized by filtration to obtain a GppNHp injection with a concentration of 5-15 μg/μL. When the GppNHp injection is administered to mice, an administration amount of 0.5-1.5 μL/g of mouse body weight and a dosing frequency of 1-5 days/time are used, with consecutive administration for 8-15 times. A concentration of the GppNHp injection is preferably 8-12 μg/μL, and more preferably 10 μg/μL. A concentration of the phosphate buffer is 0.001-0.02 M, preferably 0.005-0.015 M, and more preferably 0.01 M. A pH of the phosphate buffer is 7.0-7.6, preferably 7.1-7.5, and more preferably 7.2-7.4. Sterile filtration is performed using a filter membrane with a pore size of 0.22 μm. A use amount of GppNHp is preferably 0.8-1.2 μL/g of mouse body weight, and more preferably 1 μL/g of mouse body weight. A dosing frequency is preferably 2-4 days/time, and more preferably 3 days/time. A number of consecutive administrations is preferably 9-12 times, and more preferably 10 consecutive times.

The present invention further provides a medicament for treating systemic lupus erythematosus, where the medicament includes GppNHp and a pharmaceutically acceptable carrier thereof.

In the present invention, the medicament is preferably an injection including GppNHp.

The technical solutions provided by the present invention will be described in detail below with reference to examples, which, however, should not be construed as limiting the scope of the present invention.

### Example

### Experimental Materials and Preparation

1) Experimental animals: Male MRL/lpr mice aged 14-16 weeks were selected (purchased from Cyagen Biosciences Inc.), and these mice were widely used as disease models because of autoimmune characteristics similar to human systemic lupus erythematosus.

2) Drug preparation: GppNHp (purchased from MCE, Cat. No. HY-137167) was dissolved in phosphate-buffered saline (PBS, 0.01 M, pH 7.2-7.4) at a concentration of 10 μg/μL. All solutions were sterilized by filtration through a 0.22 μm filter membrane before use.

### Experimental Design

1) Grouping and treatment: MRL/lpr mice were randomly divided into four groups. A treatment group was intraperitoneally injected with necroptosis inhibitor GppNHp solution (MRL/lpr+GppNHp), and control groups were intraperitoneally injected with equal volumes of DMSO (MRL/lpr+DMSO) and CTX (MRL/lpr+CTX, CTX purchased from Sigma). Injections were performed once every three days, with a dose of 1 μL/g of body weight per injection, for a total of 10 consecutive injections. Male MRL/Mpj mice aged 14-16 weeks (purchased from Cyagen Biosciences Inc.) were selected as a healthy control group without any treatment.

2) Physiological parameter monitoring: During the injection period, body weight, food intake, and water intake of mice were monitored daily to evaluate potential toxicity of the drugs and effects on overall health status of the animals.

### Collection and Analysis of Biological Samples

1) Urine and blood samples: On the third day after the last injection of the experiment, urine and venous blood of mice were collected. Urine was stored at -80 °C for subsequent analysis of urinary protein concentration. Blood was added into heparin-containing tubes, left at 25 °C for 30 min, and then centrifuged at 3000 rpm and 4 °C for 15 min. Supernatant was collected for biochemical analysis.

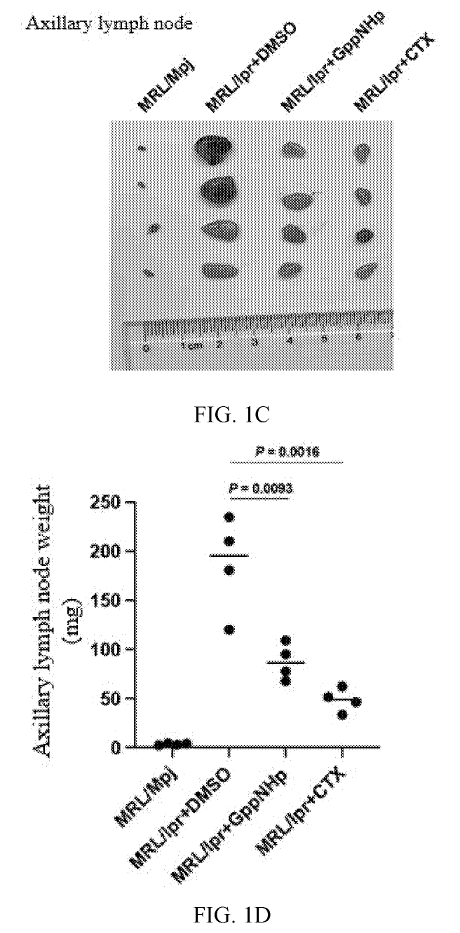

2) Tissue samples: After eyeball blood collection, mice were sacrificed, and spleen, lymph nodes, kidneys, and other relevant organs were collected for measurement of weight and volume, with results shown in FIGS. 1A-1D. All organs were fixed in 4% paraformaldehyde and then subjected to HE staining to evaluate pathological changes of the tissues, with results shown in FIG. 2.

### Biomarker analysis

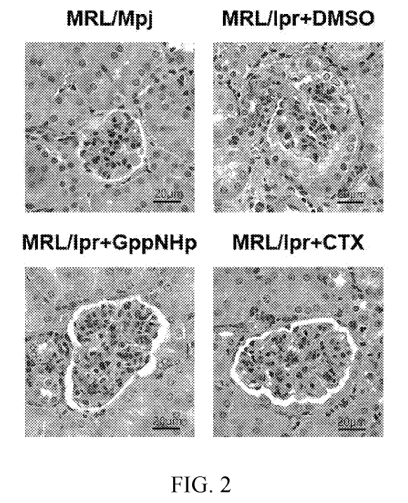

1) Immunological analysis: Levels of TNF-α, complement C3, and anti-double-stranded DNA (dsDNA) antibody in serum were determined using ELISA kits, with results shown in FIGS. 3A-3C.

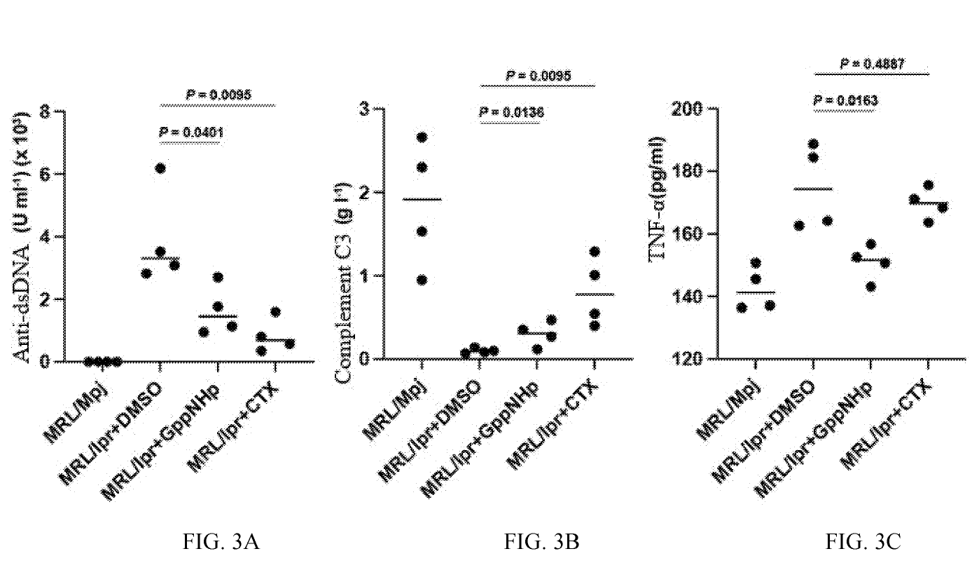

Renal function test: Serum creatinine and urinary protein concentration were measured to evaluate the extent of kidney injury, with results shown in FIGS. 4A-4B.

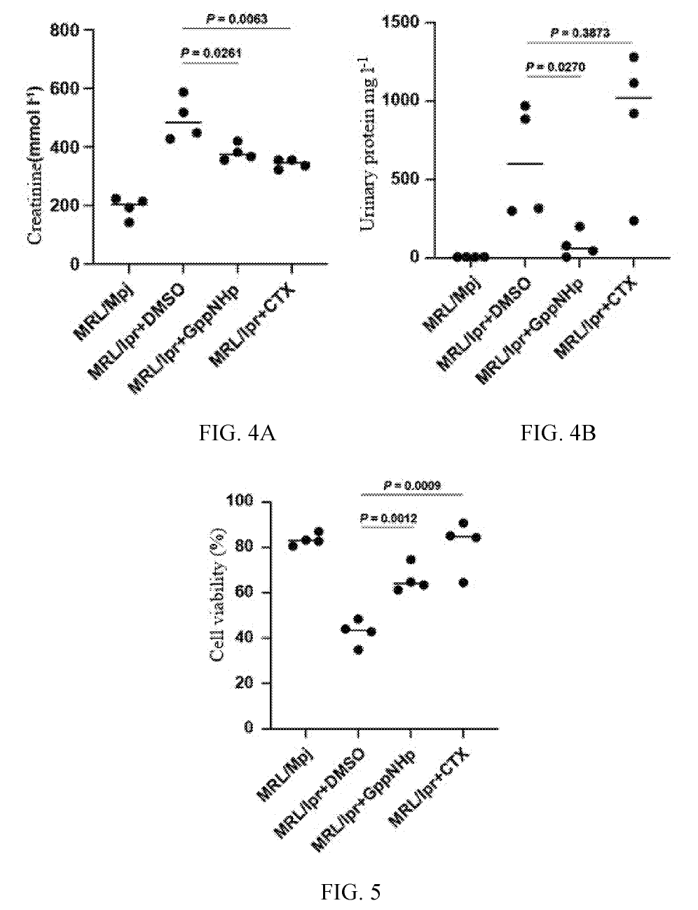

2) Flow cytometry analysis: Peripheral blood neutrophils were isolated using Miltenyi Biotec kits (purchased from Miltenyi Biotec GmbH, Germany), and cell viability was assessed, with results shown in FIG. 5.

### Data Processing and Statistical Analysis

Statistical methods: Statistical analysis was performed using GraphPad Prism 8.0 software. Measurement data were expressed as X±s, and comparisons between two groups were performed using t-test. P<0.05 indicated a statistically significant difference between the two groups.

### Experimental Results and Evaluation

As shown in FIGS. 1A-1D, compared with the MRL/lpr+DMSO control group, mice treated with GppNHp exhibited significantly reduced spleen and axillary lymph node volume and weight, indicating that GppNHp treatment enabled significant reduction of spleen and axillary lymph node volume and weight. As shown in FIG. 2, the normal mouse group (MRL/Mpj) represented a healthy control: glomerular structure was relatively intact, cells were arranged tightly, and no obvious pathological features were observed. The classical lupus model mouse group (MRL/lpr+DMSO) without receiving treatment and with DMSO as a control exhibited markedly abnormal glomerular structure, glomerular basement membrane thickening, disordered cell arrangement, and infiltration of inflammatory cells, indicating severe local pathological damage in the kidneys. The mice treated with necroptosis inhibitor GppNHp (MRL/lpr+GppNHp) showed improved glomerular structure compared with the untreated MRL/lpr+DMSO group. Basement membrane thickening was alleviated, cell arrangement was tighter, and inflammatory infiltration was reduced, indicating that GppNHp treatment exerted a certain improvement effect on pathological kidney injury. CTX (cyclophosphamide) is a commonly used immunosuppressant for clinical treatment of SLE patients. The mice treated with CTX (MRL/lpr+CTX) served as a positive control. Compared with the MRL/lpr+DMSO group, pathological kidney injury in glomeruli was significantly alleviated, and tissue structure was improved close to normal, although a small number of inflammatory cells were still observed. As shown in FIGS. 3A-5, compared with the MRL/lpr+DMSO control group, mice treated with GppNHp exhibited significantly reduced urinary protein concentration and blood creatinine concentration, indicating that GppNHp alleviated kidney injury in lupus model mice. After treatment with GppNHp, anti-dsDNA antibody concentration and tumor necrosis factor TNF-α concentration decreased, and complement C3 concentration increased. Therefore, necroptosis inhibitor GppNHp corrects abnormal autoimmune responses in lupus model mice by regulating immune cell activity, thereby significantly alleviating the lupus phenotype of mice. This provides an important theoretical and practical basis for development of new strategies for treating systemic lupus erythematosus and has broad application prospects.

The above descriptions are only preferred embodiments of the present invention. It should be noted that those of ordinary skill in the art can also make several improvements and modifications without departing from the principle of the present invention, and such improvements and modifications shall fall within the protection scope of the present invention.

What is claimed is:

1. A use of GppNHp in preparation of a medicament for treating systemic lupus erythematosus.

2. The use according to claim 1, wherein a concentration of the GppNHp is 5-15 μg/μL.

3. The use according to claim 2, wherein a solvent of the GppNHp is phosphate buffer.

4. The use according to claim 3, wherein the GppNHp is used in injection.
